(19) United States
(12) Patent Application Publication
(10) Pub. No.: US 2004/0167437 A1
(43) Pub. Date: Aug. 26, 2004

(54) Articulating intracorporal medical device

(76) Inventors: James S. Sharrow, Bloomington, MN (US); Anthony C. Vrba, Maple Grove, MN (US)

Correspondence Address: J. Scot Wickhem, CROMPTON, SEAGER & TUFTE, LLC, Suite 895, 331 Second Avenue South, Minneapolis, MN, 55401-2246, US

(21) Appl. No.: 10/375,493

(22) Filed: Feb. 26, 2003

(51) Int. Cl. A61M 25/09

(52) U.S. Cl. 600/585

(57) ABSTRACT

Intracorporal medical devices and method of making and using the same. The invention includes an intracorporal medical device having a proximal section and a distal section. An articulating member may be disposed adjacent the proximal and distal sections. The articulating member may provide the intracorporal medical device with improved bending characteristics.

### FIELD OF THE INVENTION

The invention relates to intracorporal medical devices, for example, intravascular medical devices. More particularly, the invention relates to intracorporal medical devices that include an articulating section or member, which may have desirable flexibility or bending characteristics.

### BACKGROUND

A wide variety of intracorporal medical devices have been developed for medical use, for example, intravascular use. Some of these devices include guidewires, catheters, and other such devices that have certain flexibility characteristics. Of the known intracorporal medical devices that have defined flexibility characteristics, each has certain advantages and disadvantages. There is an ongoing need to provide alternative designs and methods of making and using medical devices with desirable flexibility characteristics.

### BRIEF SUMMARY

The invention provides design, material, and manufacturing method alternatives for intracorporal medical devices having desired flexibility characteristics. In at least some embodiments, the medical devices include an elongate shaft that has a proximal portion, a distal portion, and an articulating portion and/or an articulating member that may be disposed between and adjacent the proximal and distal portions. The articulating member may be configured to provide the medical device with desirable lateral flexability or bending characteristics at a particular location along the length of the shaft.

In at least some embodiments, the articulating section is positioned at a location along the length of the medical device such that when the device is used intracorporally, the articulating section corresponds with a particular portion of the anatomy that requires the medical device to bend or flex relatively aggressively during use. For example, in some embodiments, the articulating section is positioned at a point along the length of the device such that when the distal portion of the medical device extends to a desired location within the anatomy of a patient, the articulating portion is disposed within a portion of the anatomy that requires the medical device to make a relatively aggressive bend or turn. In at lease some embodiments, the articulating portion or member can be configured to have increased lateral flexibility relative to the adjacent proximal and distal portions of the shaft, and as such, has a relatively enhanced capability to extend within an aggressive bend or turn in the anatomy. Some of the other features and characteristics of example guidewires are described in more detail below.

### BRIEF DESCRIPTION OF THE DRAWINGS

FIG. 1 is partial cross-sectional view of an example guidewire;

FIG. 2 is a partial cross-section view of another example guidewire;

FIG. 3 is a partial cross-section view of another example guidewire;

FIG. 4 is a partial cross-section view of another example guidewire;

FIG. 5 is a partial cross-section view of another example guidewire;

FIG. 6 is a partial cross-section view of another example guidewire;

FIG. 7 is a partial cross-section view of another example guidewire;

FIG. 8 is a plan view of an example guidewire disposed within a portion of the vasculature of a patient;

FIG. 9 is a plan view of an example guidewire disposed within another portion of the vasculature of a patient;

FIG. 10 is a perspective view of another example guidewire;

FIG. 11 is an end view of another example guidewire;

FIG. 12 is a partial cross-section view of the example guidewire shown in FIG. 11;

FIG. 13 is another partial cross-section view of the example guidewire shown in FIG. 11;

FIG. 14 is a side view of another example guidewire;

FIG. 15 is a side view of another example guidewire;

FIG. 16 is a partial cross-section view of another example guidewire;

FIG. 17 is a side view of an example core wire;

FIG. 18 is a side view of another example core wire; and

FIG. 19 is a side view of another example core wire.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

Weight percent, percent by weight, wt %, wt-%, % by weight, and the like are synonyms that refer to the concentration of a substance as the weight of that substance divided by the weight of the composition and multiplied by 100.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention. For example, although discussed with specific reference to guidewires in the particular embodiments described herein, the invention may be applicable to a variety of medical devices that are adapted to be advanced into the anatomy of a patient through an opening or lumen. For example, the invention may be applicable to fixed wire devices, catheters (e.g. balloon, stent delivery, etc.) drive shafts for rotational devices such as atherectomy catheters and IVUS catheters, endoscopic devices, laproscopic devices, embolic protection devices, spinal or cranial navigational or therapeutic devices, and other such devices.

Refer now to FIG. 1, which is a partial cross-sectional view of an example guidewire 10. Guidewire 10 may include a proximal section 12, a distal section 14, and an articulating section 16. As used herein, the proximal section 12 and the distal section 14 may generically refer to any two adjacent guidewire sections along any portion of the guidewire. Those of skill in the art and others will recognize that the materials, structure, and dimensions of the proximal/distal guidewire sections 12/14 are dictated primary by the desired characteristics and function of the final guidewire, and that any of a broad range of materials, structures, and dimensions can be used.

The articulating section 16 is disposed at a location along the length of the guidewire 10 between proximal section 12 and distal section 14. Articulating section 16 may be adapted or configured to have flexibility characteristics that allow it to bend or flex to form relatively tight angles. Typically, the articulating section 16 has flexibility characteristics that make it more flexible than the adjacent portions of the proximal section 12 and distal section 14 of the guidewire 10. Articulating section 16 may also be configured or adapted for not only low force bending or flexing, but also for allowing torque and push forces to transfer from proximal section 12 to distal section 14. The articulating section 16 can be positioned at a location along the length of the guidewire such that when the device is used intracorporally at a particular location in the anatomy, the articulating section 16 corresponds with a particular part of the anatomy that requires the guidewire to bend or flex relatively aggressively during use. For example, in some embodiments, the articulating section is positioned at a location along the length of the device such that when the distal portion of the guidewire extends to a desired location within the anatomy of a patient, the articulating section 16 is disposed within a portion of the anatomy that requires the guidewire to make a relatively tight or aggressive bend or turn. Some of the other features and characteristics of articulating section 16 are described in more detail below.

The guidewire 10 can include one or more shaft or core portions. For example, the proximal section 12 of guidewire 10 may include a proximal shaft member 18. Similarly, distal section 14 may include a distal shaft member 20. The shaft members 18/20 may be distinct structures that can be connected or attached to one another and/or may be connected, but longitudinally spaced from each other, for example a distance D as shown in FIG. 1. Distance D can vary and may be in the range of about 10 centimeters or less. In some embodiments, the space defined by distance D may be left empty. Alternatively, the space may be filled with an appropriate material, for example, connector or binding material, radiopaque material, or the like. Alternatively, the central shaft or core portion can be one continuous member. For example, the proximal shaft member 18 and distal shaft member 20 may be continuous with one another and, collectively, define a continuous shaft or core. However, in such embodiments, the shaft or core portion includes a section within the articulating section 16 that includes increased flexability characteristics. Such increased flexability characteristics can be achieved through varying the material or structure of the shaft, as discussed in more detail below.

Shaft members 18/20 (in embodiments where shaft members 18/20 define a continuous core wire and in embodiments where shaft members 18/20 are distinct structures) may include metals, metal alloys, polymers, or the like, or combinations or mixtures thereof Some examples of suitable metals and metal alloys include stainless steel, such as 304V, 304L, and 316L stainless steel; alloys including nickel-titanium alloy such as linear elastic or superelastic (i.e. pseudoelastic) nitinol; nickel-chromium alloy; nickel-chromium-iron alloy; cobalt alloy; tungsten or tungsten alloys; MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); hastelloy; monel 400; inconel 825; or the like; or other suitable material.

The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL).

Within the family of commercially available nitinol alloys, is a category designated “linear elastic” which, although is similar in chemistry to conventional shape memory and superelastic (i.e. pseudoelastic) varieties, exhibits distinct and useful mechanical properties. Some examples of these and other properties can be found in U.S. Pat. Nos. 5,238,004 and 6,508,803, which are herein incorporated by reference. By skilled applications of cold work, directional stress, and heat treatment, the wire is fabricated in such a way that it does not display a substantial “superelastic plateau” or “flag region” in its stress/strain curve. Instead, as recoverable strain increases, the stress continues to increase in an essentially linear relationship until plastic deformation begins. In some embodiments, the linear elastic nickel-titanium alloy is an alloy that does not show any martensite/austenite phase changes that are detectable by DSC and DMTA analysis over a large temperature range.

For example, in some embodiments, there is no martensite/austenite phase changes detectable by DSC and DMTA analysis in the range of about −60° C. to about 120° C. The mechanical bending properties of such material are therefore generally inert to the effect of temperature over this very broad range of temperature. In some particular embodiments, the mechanical properties of the alloy at ambient or room temperature are substantially the same as the mechanical properties at body temperature. In some embodiments, the use of the linear elastic nickel-titanium alloy allows the guidewire to exhibit superior “pushability” around tortuous anatomy.

In some embodiments, the linear elastic nickel-titanium alloy is in the range of about 50 to about 60 weight percent nickel, with the remainder being essentially titanium. In some particular embodiments, the composition is in the range of about 54 to about 57 weight percent nickel. One example of a suitable nickel-titanium alloy is FHP-NT alloy commercially available from Furukawa Techno Material Co. of Kanagawa, Japan. In some other embodiments, a superelastic alloy, for example a superelastic nitinol can be used to achieve desired properties.

In at least some embodiments, portions or all of shaft members 18/20, or other structures included within the guidewire 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of guidewire 10 in determining its location. Some examples of radiopaque materials can include, but are not limited to, gold, platinum, palladium, tantalum, tungsten alloy, polymer material loaded with a radiopaque filler, and the like. Additionally one or more radiopaque marker members 21 (e.g., marker bands, marker coils, and the like) may be disposed adjacent articulating section 16 and/or the articulating member 24.

In some embodiments, a degree of MRI compatibility is imparted into guidewire 10. For example, to enhance compatibility with Magnetic Resonance Imaging (MRI) machines, it may be desirable to make shaft members 18/20, or other portions of guidewire 10, in a manner that would impart a degree of MRI compatibility. For example, shaft members 18/20, or portions thereof, may be made of a material that does not substantially distort the image and create substantial artifacts (artifacts are gaps in the image). Certain ferromagnetic materials, for example, may not be suitable because they may create artifacts in an MRI image. Shaft members 18/20, or portions thereof, may also be made from a material that the MRI machine can image. Some materials that exhibit these characteristics include, for example, tungsten, Elgiloy, MP35N, nitinol, and the like, and others.

As stated above, shaft members 18/20 can be made of the same material, or in some embodiments, can include portions or sections made of different materials. In some embodiments, the material used to construct guidewire 10 is chosen to impart varying flexibility and stiffness characteristics to different portions of guidewire 10. For example, proximal shaft member 18 and distal shaft member 20 may be formed of different materials, for example materials having different moduli of elasticity, resulting in a difference in flexibility. In some embodiments, the material used to construct proximal shaft member 18 can be relatively stiff for pushability and torqueability, and the material used to construct distal shaft member 20 can be relatively flexible by comparison for better lateral trackability and steerability. For example, proximal shaft member 18 can be formed of straightened 304v stainless steel wire or ribbon, and distal shaft member 20 can be formed of a straightened super elastic or linear elastic alloy, for example a nickel-titanium alloy wire or ribbon.

The length of shaft members 18/20 (and/or the length of guidewire 10) are typically dictated by the length and flexibility characteristics desired in the final medical device. For example, proximal section 12 may have a length in the range of about 20 to about 300 centimeters or more and distal section 14 may have a length in the range of about 3 to about 50 centimeters or more. It can be appreciated that alterations in the length of sections 12/14 can be made without departing from the spirit of the invention.

Shaft members 18/20 can have a solid cross-section, but in some embodiments, can have a hollow cross-section. In yet other embodiments, shaft members 18/20 can include combinations of areas having solid cross-sections and hollow cross sections. Moreover, shaft members 18/20 can be made of rounded wire, flattened ribbon, or other such structures having various cross-sectional geometries. The cross-sectional geometries along the length of shaft members 18/20 can also be constant or can vary. For example, FIG. 1 depicts shaft members 18/20 as having a round cross-sectional shape. It can be appreciated that other cross-sectional shapes or combinations of shapes may be utilized without departing from the spirit of the invention. For example, the cross-sectional shape of shaft members 18/20 may be oval, rectangular, square, polygonal, and the like, or any suitable shape.

As shown in FIG. 1, distal shaft member 20 may include one or more tapers or tapered regions. In some embodiments distal shaft member 20 may be tapered and have an initial outside size or diameter that can be substantially the same as the outside diameter of proximal shaft member 18, which then tapers to a reduced size or diameter. For example, in some embodiments, distal shaft member 20 can have an initial outside diameter that is in the range of about 0.010 to about 0.020 inches that tapers to a diameter in the range of about 0.001 to about 0.005 inches. The tapered regions may be linearly tapered, tapered in a curvilinear fashion, uniformly tapered, non-uniformly tapered, or tapered in a step-wise fashion. The angle of any such tapers can vary, depending upon the desired flexibility characteristics. The length of the taper may be selected to obtain a more (longer length) or less (shorter length) gradual transition in stiffness. Although FIG. 1 depicts distal shaft member 20 as being tapered, it can be appreciated that essentially any portion of guidewire 10 and/or shaft members 18/20 may be tapered and the taper can be in either the proximal or the distal direction. As shown in FIG. 1, the tapered region may include one or more portions where the outside diameter is narrowing, for example, the tapered portions, and portions where the outside diameter remains essentially constant, for example, constant diameter portions. The number, arrangement, size, and length of the narrowing and constant diameter portions can be varied to achieve the desired characteristics, such as flexibility and torque transmission characteristics. The narrowing and constant diameter portions as shown in FIG. 1 are not intended to be limiting, and alterations of this arrangement can be made without departing from the spirit of the invention.

The tapered and constant diameter portions of the tapered region may be formed by any one of a number of different techniques, for example, by centerless grinding methods, stamping methods, and the like. The centerless grinding technique may utilize an indexing system employing sensors (e.g., optical/reflective, magnetic) to avoid excessive grinding of the connection. In addition, the centerless grinding technique may utilize a CBN or diamond abrasive grinding wheel that is well shaped and dressed to avoid grabbing core wire during the grinding process. In some embodiments, distal shaft member 20 can be centerless ground using a Royal Master HI-AC centerless grinder.

As indicated above, the articulating section 16 is disposed at a location along the length of the guidewire 10 between proximal section 12 and distal section 14, and is adapted or configured to have flexibility characteristics that allows it to have an increased ability to bend or laterally flex to form relatively tight angles relative to the adjacent portions of the proximal section 12 and distal section 14. Typically, the articulating section 16 has flexibility characteristics that make it more laterally flexible than the adjacent portions of the proximal section 12 and distal section 14 of the guidewire 10. Those of skill in the art and others will recognize that the materials, structure, and dimensions of the articulating section 16 are dictated primary by the desired flexibility characteristics and function of the final guidewire, and that any of a broad range of materials, structures, and dimensions can be used.

In at least some embodiments, articulating section 16 may include or be defined by an articulating member 24. Articulating member 24 may be made from any appropriate structure and material including any of those described herein. In some embodiments, the articulating member 24 may be generally tubular so that it can couple a distal end 26 of proximal shaft member 18 and a proximal end 28 of distal shaft member 20. According to this embodiment, distal end 26 of proximal shaft member 18 and proximal end 28 of distal shaft member 20 may be disposed in opposite ends of the tubular articulating member 24. Ends 26/28 may be loosely disposed within articulating member 24 or ends 26/28 may be secured to articulating member 24. Securing may be achieved in a number of ways. For example, ends 26/28 may be secured to articulating member 24 by friction fitting, mechanically fitting, chemically bonding, thermally bonding, welding (e.g., resistance or laser welding), soldering, brazing, adhesive, the use of an outer sleeve or polymer layer to bond or connect the components, or the like, or combinations thereof. Some examples of suitable connection techniques are also disclosed in U.S. patent application Ser. Nos. 09/972,276, and 10/086,992, which are incorporated herein by reference. Additionally, in some embodiments, ends 26/28 may be secured to articulating member 24 by using an expandable alloy, for example a bismuth alloy. Some examples of methods, techniques and structures that can be used to interconnect different portions of a guidewire using such expandable materials are disclosed in a U.S. Patent Application entitled “Composite Medical Device” (Attorney docket number 1001.1546101) filed on even date with this application and which is hereby incorporated by reference.

FIG. 1 illustrates a plurality of bonding points 32, which may comprise any of the bonding or securing means described herein, disposed adjacent ends 26/28 and articulating member 24.

Lateral flexibility, bendability or other such characteristics of the articulating member 24 can be achieved or enhanced in a number of ways. For example, the materials selected for articulating member 24 may be chosen so that articulating section 16 has a greater lateral flexibility than the lateral flexibilities of proximal shaft member 18 adjacent distal end 26 and distal shaft member 20 adjacent proximal end 28. For example, articulating section 16 may be formed of materials having a different modulus of elasticity than the adjacent portions of the proximal shaft member 18 and distal shaft member 20, resulting in a difference in flexibility. Alternatively, articulating member 24 may include a thin wall tubular structure, made from essentially any appropriate material including those described herein, having desirable lateral flexibility characteristics.

In addition to, or as an alternative to material composition, the desired lateral flexability or bending characteristics can be imparted or enhanced by the structure of the articulating member 26. For example, a plurality of grooves, cuts, slits, or slots 30 can be formed in a tubular articulating member 24. Such structure may be desirable because they may allow articulating member 24 to be bendable as well as transmit torque and pushing forces from proximal section 12 to distal section 14. The cuts or slots or grooves 30 can be formed in essentially any known way. For example, cuts 30 can be formed by mechanical methods, such as micro machining, saw cutting, laser cutting, chemically etching, treating or milling, casting, molding, other known methods, and the like. In some embodiments, cuts or slots 30 can completely penetrate articulating member 24. In other embodiments, cuts or slots 30 may only partially extend into articulating member 24, or include combinations of both complete and partial cuts. In some embodiments, an elastic or low modulus filler material may be disposed within slots 30 to keep coating or sheath materials, such as the sheath 22, from filling in slots 30 and, possibly, reducing the flexibility of articulating member 24.

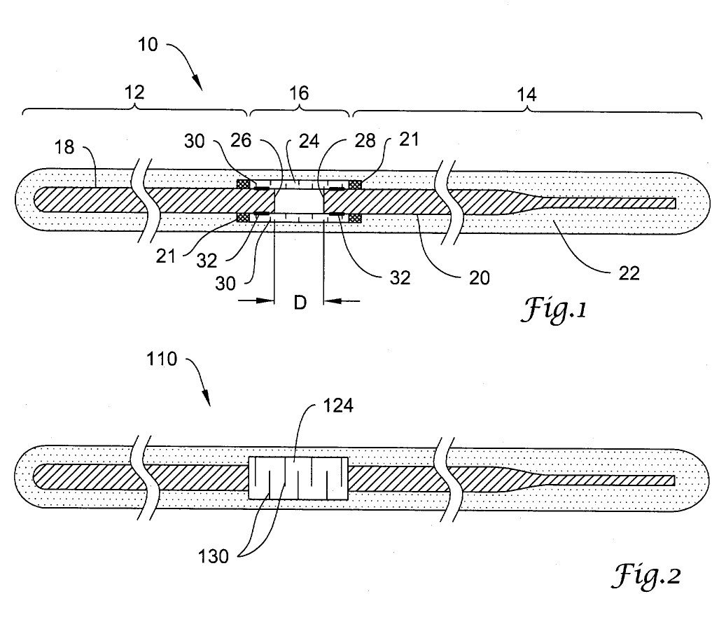

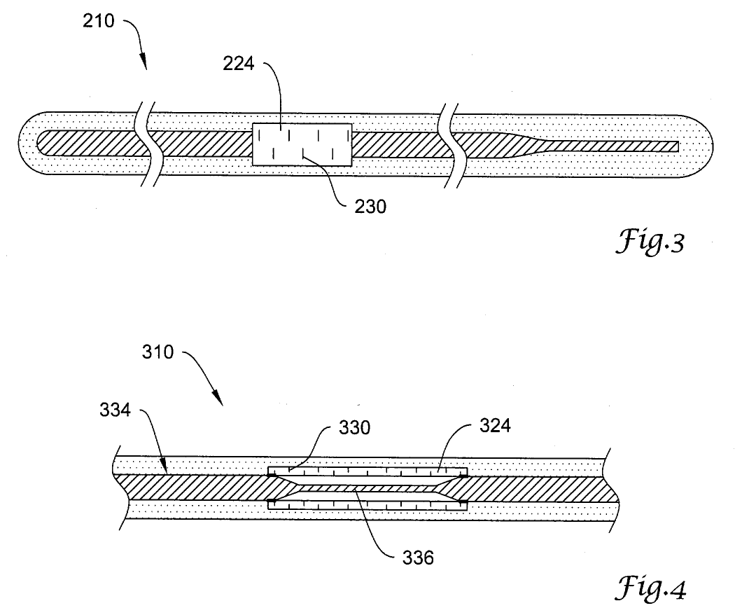

The arrangement of the cuts or slots 30 may vary. For example, the cuts or slots 30 may be formed such that one or more spines or beams are formed in the tubular member. Such spines or beams could include portions of the tubular member that remain after the cuts or slots are formed in the body of the tubular member. Such spines or beams can act to maintain a relatively high degree of tortional stiffness, while maintaining a desired level of lateral flexibility. In some embodiments, some adjacent cuts or slots can be formed such that they include portions that overlap with each other about the circumference of the tube. For example, FIG. 2 is a partial cross-sectional view of another example guidewire 110 that includes slots 130 disposed in an overlapping pattern. In other embodiments, some adjacent slots or cuts can be disposed such that they do not necessarily overlap with each other, but are disposed in a pattern that provides the desired degree of lateral flexibility. For example, FIG. 3 is a partial cross-sectional view of guidewire 210 that includes articulating member 224 including non-overlapping or opposing slots 230.

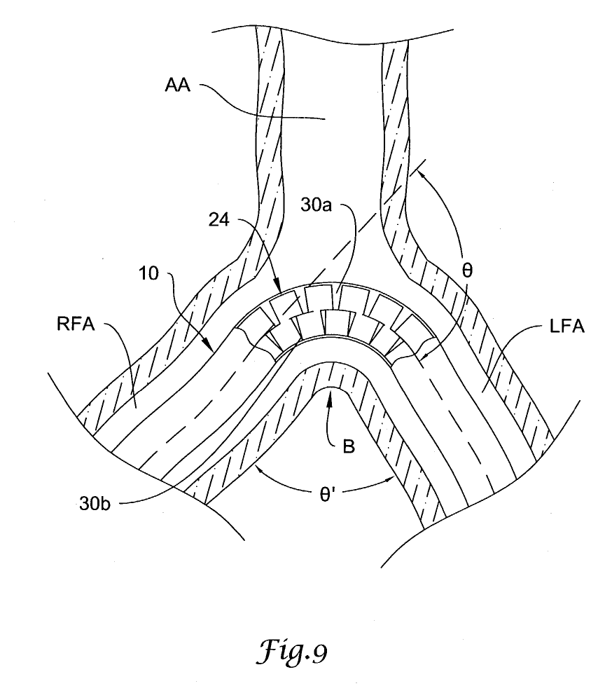

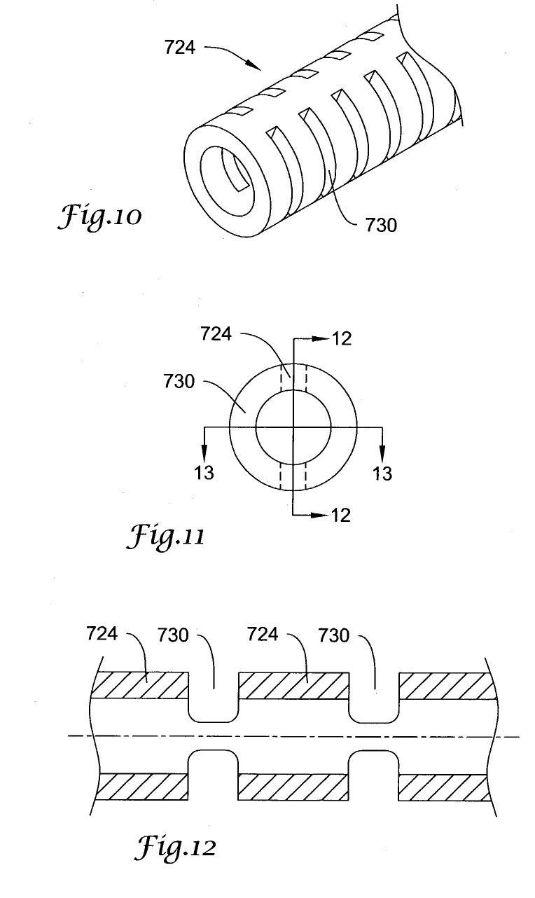

A number of additional variations in shape, arrangement, and pattern may be used. For example, another example articulating member 724, suitable for use with any of the devices described herein, is shown in FIG. 10. Articulating member 724 is similar to others described herein, except that slots 730 are rectangular in shape or pill-shaped, span nearly 180 degrees around articulating member 724, and are essentially disposed on opposite sides of articulating member 724. This figure illustrates a number of features of this and other articulating members. For example, the shape of slots 730 can vary to include essentially any appropriate shape. This may include having an elongated shape, rounded or squared edges, variability in width, and the like. Additionally, FIG. 10 illustrates that slots 730 may be arranged in a symmetrical pattern, such as being disposed essentially equally on opposite sides of articulating member 724, or in a non-symmetric or irregular pattern.

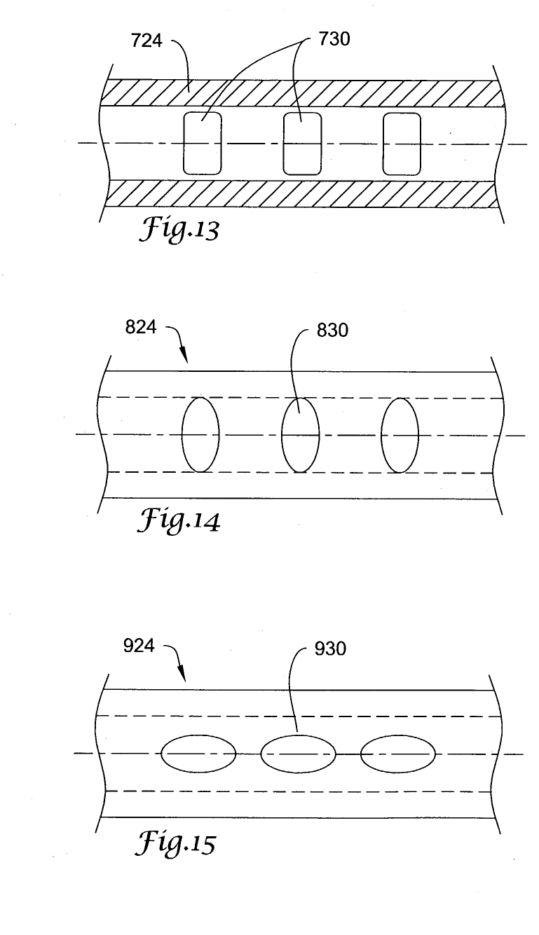

An end view of articulating member 724 is shown in FIG. 11. FIG. 11 shows the uncut areas of articulating member 724 (indicated by reference number 724) and the cut or slotted areas 730. Again, this figure illustrates that slots 730 may have a length that spans a significant portion of the circumference of articulating member 724, for example, approximating 180 degrees. For example, slots 730 may span about 175 degrees or less, 160 degrees or less, 145 degrees or less, 120 degrees or less, etc. The pattern of slots 730 can be observed by comparing FIG. 12 (which is a cross-sectional view taken through line 12-12 in FIG. 11) with FIG. 13 (which is a cross-sectional view taken through line 13-13 in FIG. 11).

Additionally, the size, shape, spacing, or orientation of the cuts or slots, or in some embodiments, the associated spines or beams, can be varied to achieve the desired lateral flexibility and/or tortional rigidity characteristics of the articulating member. Some examples of suitable shapes include squared, round, rectangular, oval, polygonal, irregular, and the like, or any other suitable shape. For example, FIG. 14 is a side of an example articulating member 824 having a plurality of oval slots 830. Similar to what is described above, the arrangement of slots 824 may vary. For example, FIG. 14 illustrates slots 830 arranged as a series of vertical ovals aligned side-by-side. Alternatively, FIG. 15 illustrates another example articulating member 924 with oval slots 930 arranged as a series of horizontal ovals aligned side-by-side. A number of addition arrangements may also be used. For example, the slots can be axially aligned, staggered, irregularly disposed, disposed either longitudinally or circumferentially (or both) about articulating member 824, or otherwise be in any suitable arrangement.

The number or density of the cuts or slots along the length of the articulating member may also vary, depending upon the desired characteristics. For example, the number or proximity of slots to one another near the midpoint of the length of the articulating member 24 may be high, while the number or proximity of slots to one another near either the distal or proximal end of the articulating member, or both, may be relatively low, or vice versa. Collectively, these figures and this description illustrate that changes in the arrangement, number, and configuration of slots may vary without departing from the scope of the invention. Some additional examples of arrangements of cuts or slots formed in a tubular body are disclosed in U.S. Pat. No. 6,428,489 and in Published U.S. patent application Ser. No. 09/746,738 (Pub. No. US 2002/0013540), both of which are incorporated herein by reference.

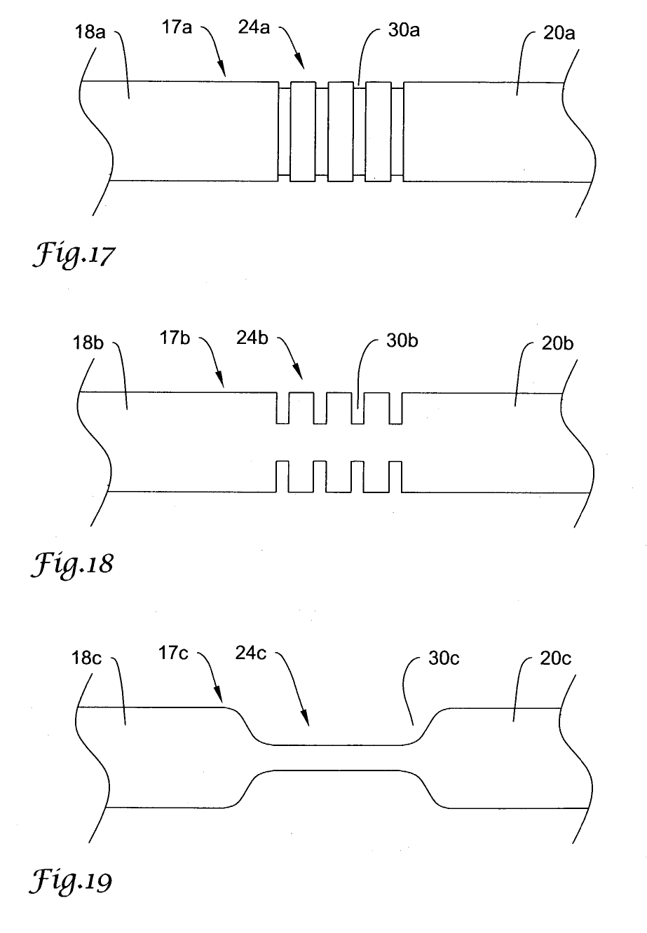

In other embodiments, the articulating section may include other structure to provide the desired increase in lateral flexibility. For example, the articulating section may include a hinge-like structure, for example a ball and socket type hinge, may include structural narrowing of all or portions of the guidewire shaft within the articulating region, may include cuts, slots, or grooves defined in the outer surface of the core wire or shaft, or other such structure. For example, FIG. 17 shows a plurality of grooves 30a formed in the outer surface of the core wire 17a at articulating section 24a. Similar to other core wires described herein, core wire 17a may include proximal section 18a and distal section 20a. Additionally, FIG. 18 shows a plurality of slots 30b formed in the outer surface of core wire 17b (including proximal section 18b and distal section 20b) at articulating section 24b. Moreover, FIG. 19 shows a necked-down or narrowing slot 30c defining articulating section 24c of core wire 17c (including proximal section 18c and distal section 20c).

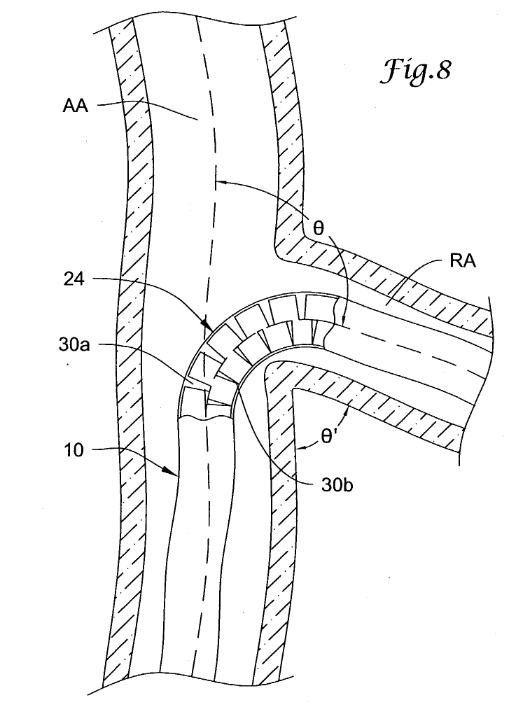

As stated above, the position of articulating section 16 can vary depending on the intended use of the guidewire 10. For example, uses of guidewire 10 may include navigating guidewire 10 across aggressive intravascular bends or curves in order to reach a target site or area. According to these embodiments, it may be desirable to position articulating section 16 so that it can correspond to these curves or bends when the distal region of the guidewire 10 is disposed adjacent the target site. For example, the vasculature may bend or curve such that guidewire 10 may need to bend 45 degrees or more, 60 degrees or more, 90 degrees or more, 120 degrees or more, etc. in order to navigate, span, or otherwise extend through the curve. As such, the articulating section 16 can be located at the appropriate position along the length of guidewire 10 so that articulating section 16 can be disposed within the bend when the distal guidewire section is located adjacent the target site. Articulating section 16, thus, enhances the ability of guidewire 10 to bend or laterally flex in accordance with the requirements of the anatomy being navigated. It should be noted that the above angles of guidewire 10 bending are understood to be angles that describe the change in course of the guidewire 10 and are shown in FIG. 8 as bending angle θ. As such, when the sharpness, tightness, and aggressiveness of the intravascular bend increases, the bending angle θ of the guidewire 10 increases.

Locating the articulating section 16 along the length of the guidewire in such a manner can be advantageous in maintaining the desired position of the guidewire, for example, the position of the distal portion of the guidewire relative to a target site. In at lease some conventional guidewire constructions that do not include an articulating section, the force necessary to bend the guidewire within an aggressive turn or bend in the anatomy results in a relatively high level of stress (i.e. tension and compressive forces) being produced in the guidewire shaft at the bending point. This stress can have adverse effects upon the ability of an operator to maintain the position of portions of the guidewire, for example, the distal tip at a desired location in the anatomy. For example, tortional rotation of the guidewire may cause the tip to move, or “whip” due to the stress. Additionally, the guidewire may have a greater tendancy to slip or displace, for example, when the guidewire is rotated, or when catheter exchanges or other procedures are carried out that may place some additional force or movement on the guidewire. However, if an articulating section, as explained herein, is positioned along the length of the guidewire such that it is located within the aggressive turn or bend in the anatomy, the amount of stress can be reduced. As such, the desired positioning of the guidewire can be better maintained, for example, even during tortional rotation.

The particular distance of the location of the articulating member 24 from either the distal or proximal end of the guidewire can vary, depending upon, for example, the size or length of the anatomy of a patient, the particular location of the treatment site relative to the aggressive bend or turn in the anatomy, the lengths of the distal or proximal shaft members 18/20, and the like. Therefore, an entire series of devices is contemplated, each having one or more articulating members 24 being appropriately located along the length of the guidewire based upon the particular procedure being conducted and the particular anatomy of a patient.

One example of anatomy that can be navigated using a guidewire, but includes an aggressive bend or turn is the junction of the renal artery and the abdominal aorta in a human patient. The junction of the renal artery and the abdominal aorta may form a relatively aggressive angle, for example, an angle of about 90 degrees or more or less, when being approached from a femoral access point. A target site for treatment or navigation may be in a location adjacent to or within a renal artery or a kidney of a patient. Because of the angle formed in the anatomy at the junction of the renal artery and the abdominal aorta, it may be difficult for a distal portion of a medical device to maintain its position adjacent the target site while a portion of the wire must make the aggressive turn from the aorta to the renal artery. For example, in at lease some conventional guidewire constructions that do not include an articulating section, the force necessary to bend the guidewire within the turn in the anatomy may result in a relatively high level of residual stress in the guidewire shaft at the bending point. Thus, it may be desirable to use a guidewire including an articulating member 24 that is disposed at a location along the length of the guidewire such that when the distal portion of the guidewire is positioned at a desired location within or adjacent the target site, the articulating member 24 is positioned within the junction of the renal artery and the abdominal aorta. By including the articulating section 16 at such a location, the guidewire 10 can more easily access the renal artery when approached from a lower vascular region such as the femoral artery, and the amount of residual stress can be reduced.

In some such embodiments, the articulating section 16 can be disposed along the length of the guidewire at a location that is in the range of about 5 to about 25 centimeters from the distal end of guidewire 10. Of course the exact position can vary greatly as discussed above.

Another example of navigable anatomy that includes a relatively aggressive bend or turn is the aortic bifurcation at the base of the abdominal aorta. This is the point in the anatomy where the abdominal aorta splits and connects to the left and right femoral arteries. In some operations, it is desirable to gain access to one of the femoral arteries via a vascular access point in the other femoral artery. This requires that the guidewire (or other device) extends from one femoral artery to the other through the aortic bifurcation, which may form an angle of about 90 degrees or more or less when extending from one femoral artery to the other. Again, it may be desirable to use a guidewire including an articulating member 24 that is disposed at a location along the length of the guidewire such that when the distal portion of the guidewire is positioned at a desired location within or adjacent the target site, the articulating member 24 is positioned within the aortic bifurcation. By including the articulating section 16 at such a location, the guidewire 10 can more easily span the angle presented by the aortic bifurcation, and the desired positioning of the guidewire, for example the guidewire tip, can be better maintained. In some such embodiments, the articulating section 16 can be disposed along the length of the guidewire at a location that is in the range of about 20 to about 90 centimeters from the distal end of guidewire 10.

In some embodiments, the articulating member 24 may be generally described as being near the middle or the proximal end of guidewire 10. In other embodiments, the articulating member 24 may be generally described as being near the distal end of guidewire 10. Of course the exact position can vary greatly. According to these embodiments, guidewire 10 may include articulating member 24 disposed at other (including essentially any) position along guidewire 10.

FIG. 1 also illustrates that a coating or sheath 22 may be disposed over shaft members 18/20 and/or articulating member 16. In at least some embodiments, sheath 22 may be made from a polymer. However, any of the materials described herein may be appropriate. Some examples of suitable polymers may include polytetrafluroethylene (PTFE), fluorinated ethylene propylene (FEP), polyurethane, polypropylene (PP), polyvinylchloride (PVC), polyoxymethylene (POM), polybutylene terephthalate (PBT), polyphenylene sulfide (PPS), polyphenylene oxide (PPO), polysulfone, perfluroo (propyl vinyl ether) (PFA), polyether-ester (for example a polyether-ester elastomer such as ARNITEL® available from DSM Engineering Plastics), polyester (for example a polyester elastomer such as HYTREL® available from DuPont), polyamide (for example, DURETHAN® available from Bayer or CRISTAMID® available from Elf Atochem), elastomeric polyamides, block polyamide/ethers, polyether block ester, polyether block amide (PEBA, for example available under the trade name PEBAX®), silicones, polyethylene, Marlex high-density polyethylene, linear low density polyethylene (for example REXELL®), polyolefin, polyetheretherketone (PEEK), polyimide (PI), polyetherimide (PEI), nylon, other suitable materials, or mixtures, combinations, copolymers thereof, polymer/metal composites, lubricous polymers, and the like. In some embodiments sheath 22 can include a liquid crystal polymer (LCP) blended with other polymers to enhance torqueability. For example, the mixture can contain up to about 5% LCP. This has been found to enhance torqueability.

In some embodiments, sheath 22 is disposed over essentially the entire length of guidewire 10. This may include extending distally beyond distal shaft member 20. Sheath 22 may be disposed over shaft members 18/20 and/or articulating member 24 in any one of a number of different manners. For example, sheath 22 may be disposed by thermal bonding techniques, by coating, by extrusion, co-extrusion, interrupted layer co-extrusion (ILC), or fusing several segments end-to-end. The layer or layers may have a uniform stiffness or a gradual reduction in stiffness from the proximal end to the distal end thereof. The gradual reduction in stiffness may be continuous as by ILC or may be stepped as by fusing together separate extruded tubular segments. Sheath 22 may be impregnated with a radiopaque filler material to facilitate radiographic visualization. Those skilled in the art will recognize that these materials can vary widely without deviating from the scope of the present invention.

In some embodiments, wherein the sheath 22 is disposed over the articulating section 16, it may be desirable that the sheath is disposed in such a manner that the structure within the articulating section 16, for example, an articulating member 24, can still flex or bend in an acceptable manner. For example, the portion of the sheath 22 that extends over the articulating section 16 can be made of a suitably flexible material. Additionally, in some embodiments, the sheath 22 may extend over the articulating member 24, but is not directly attached thereto, such that, for example, the slots or grooves in the articulating member can flex and move within the sheath as it flexes or bends.

In some embodiments, one or more second coating or sheath (not shown), for example a lubricious, a hydrophilic, a hydrophobic, a protective, or other type of coating may be applied over portions or all of sheath 22 and/or guidewire 10. Hydrophobic coatings such as fluoropolymers provide a dry lubricity which can improve guidewire handling and device exchanges. Lubricious coatings can also improve steerability and lesion crossing capability. Suitable lubricious polymers are well known in the art and may include silicone and the like, hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference. It may be desirable to include a plurality of different second coating, for example having different properties or lubricities. For example, it may be desirable to include a more lubricous second coating the distal end of guidewire 10 and a less lubricious second coating (which may aid the ability of the clinician to grasp guidewire 10) near the proximal end of guidewire 10.

FIG. 4 illustrates another example guidewire 310. Guidewire 310 is similar to other guidewires described herein except that it shows an example configuration where proximal and distal shaft members define a core wire 334. Core wire 334 may include a narrowed or tapered articulating section 336 (generally disposed at an articulating section of guidewire 310 that is positioned similar to articulating section 16 of guidewire 10 in FIG. 1), disposed between continuous proximal and distal shaft members 318/320. Tapered articulating section 336 may be formed according to a number of different techniques such as grinding methods described herein and others. Similar to what is described above, articulating member 324 may include one or more cuts or slots 330.

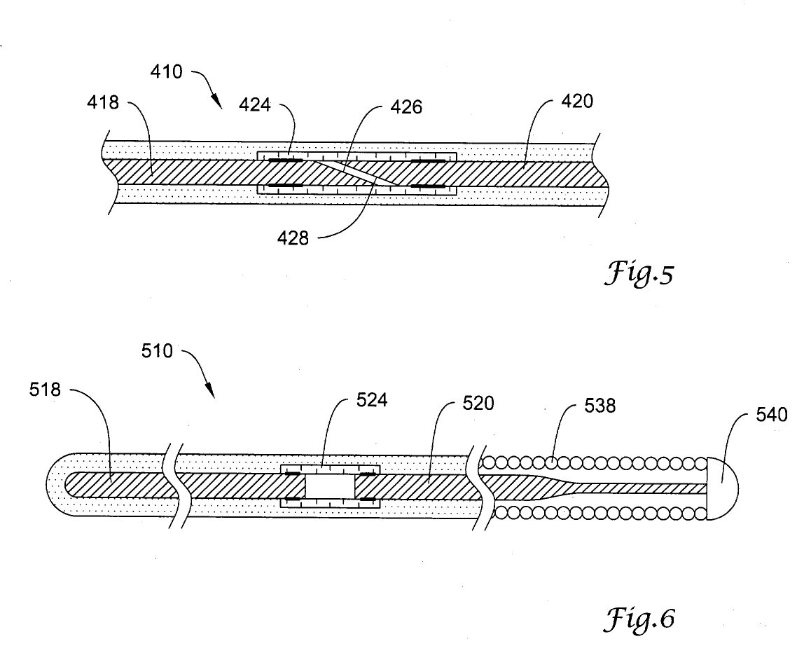

Another example guidewire 410 is shown in FIG. 5. Guidewire 410 is similar to other guidewires disclosed herein except that distal end 426 of proximal shaft member 418 and proximal end 428 of distal shaft member 420 may be angled. Articulating member 424 may be disposed over ends 426/428. In at least some embodiments, a portion of proximal and distal shaft members 418/420 may overlap. This may allow any transitions in flexibilities between shaft members 418/420 to be more gradual or smooth.

FIG. 6 is a partial cross-sectional view of another example guidewire 510. Guidewire 510 is similar to other guidewires described herein except that it includes a spring tip characterized by a distal coil 538 and a distal tip 540. Guidewire 510 may also include proximal shaft member 518, distal shaft member 520, and articulating member 524. It can be appreciated that a number of other types of guidewire tips (for example, shapeable tips, other atraumatic tips, and the like) are known in the art and may be used with any of the guidewire described herein without departing from the spirit of the invention.

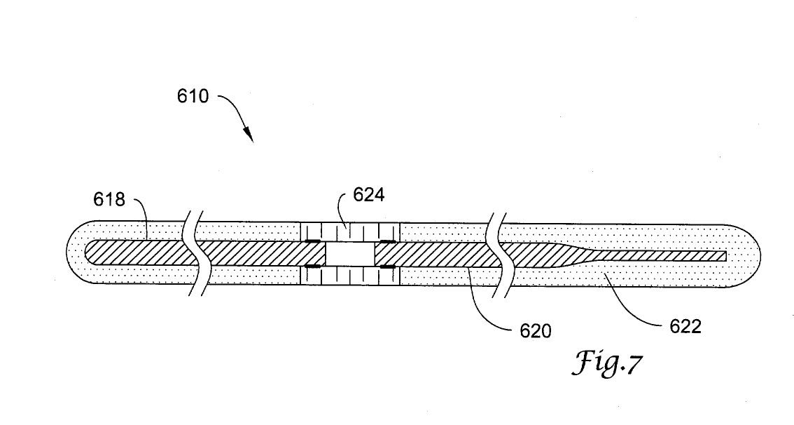

FIG. 7 is a partial cross-sectional view of another example guidewire 610. Guidewire 610 is similar to other guidewires described herein except that it articulating member 624, coupling shaft members 618/620, and sheath 622 are aligned so that at least a portion of articulating member 624 is not covered by the sheath 622.

FIG. 8 illustrates an example plan view of the use of guidewire 10 (that is similarly applicable to any of the guidewire disclosed herein) with articulating member 24 spanning the transition between the abdominal aorta AA and the renal artery RA. The renal artery RA may be disposed at an angle θ′ relative to the abdominal aorta AA. In order to span the transition, the guidewire 10 may need to bend at an angle θ, which may be in the range of about 45 degrees or greater, 60 degrees or greater, 90 degrees or greater, 120 degrees or greater, etc. The features, characteristics, and benefits of guidewire 10 may be utilized at other intravascular locations including, for example, peripheral intravascular locations as well as cardiac locations. For example, it may be desirable to dispose articulating member 24 at branching point or fork where abdominal aorta AA splits to the left and right femoral arteries.

Because angle θ′, as it can be seen, may be about ninety degrees or more or less, articulating member 24 may act as a hinge or elbow that spans the relevant transition point that may, for example, allow guidewire 10 to better hold its position while still maintaining its ability to transmit torque and other forces. It can also be seen in FIG. 8 that slots 30 within articulating member 24 may be able to alter their position when bending across a transition point. For example, FIG. 8 illustrates that some of slots 30, indicated by reference number 30a, may be opened or widened while others, indicated by reference number 30b, may be closed or narrowed. The opening or narrowing of the slots indicate that the articulating member can be adapted or configured to compensate for the tensional and compressive forces that are being placed on the articulating member as it spans the bend or turn in the anatomy.

FIG. 9 similarly illustrates another example plan view of the use of guidewire 10, or any of the other guidewires described herein, with articulating member 24 is disposed adjacent the bifurcation B where the abdominal aorta AA splits into the left femoral artery LFA and the right femoral artery RFA. According to this embodiment, guidewire 10 can be used to access one femoral artery, for example the left femoral artery LFA, by advancing guidewire 10 from the right femoral artery RFA, across the bifurcation B in the abdominal aorta AA, and into the left femoral artery LFA. Similar to what is described above, right femoral artery RFA and left femoral artery LFA may be disposed at angle θ′ relative to each other, which may be about 90 degrees or less. Accordingly, guidewire 10 may need to bend at an angle θ,which may be in the range of about 45 degrees or greater, 60 degrees or greater, 90 degrees or greater, 120 degrees or greater, etc.

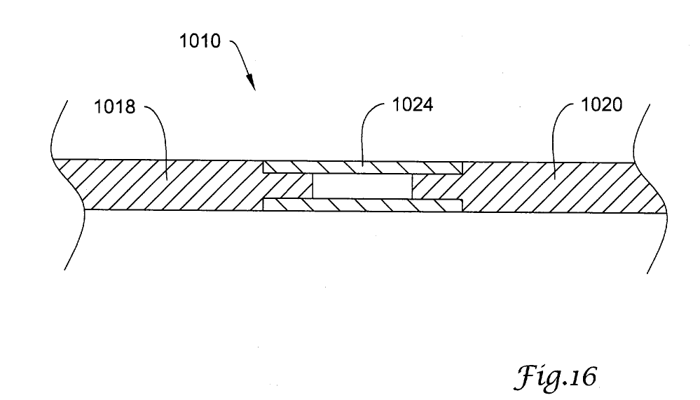

FIG. 16 illustrates another example guidewire 1010. Guidewire 1010 is similar to other guidewires described herein. For example, guidewire 1010 may include proximal shaft member 1018, distal shaft member 1020, and articulating member 1024. However, proximal and distal shaft members 1018/1020 may be stepped or necked down so that articulating member 1024 can be disposed over the ends thereof. Accordingly, guidewire 1010 may have a smooth outer surface, defined by shaft members 1018/1020 and articulating member 1024, and may not need to include an outer sheath.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

What is claimed is:

1. An elongated intracorporal medical device adapted for accessing an intracorporal target area in an anatomy of a patient, the anatomy of the patient including a first region and a second region connected by a transition region that forms a bend in the anatomy that creates an angle of sixty degrees or greater, the medical device comprising:
a proximal section configured to extend from outside the patient to within the first region in the anatomy of the patient;
a distal section configured to extend within the second region in the anatomy of the patient and to the target area within the anatomy of the patient; and
an articulating section disposed between the proximal section and the distal section, the articulating section being defined by an articulating member disposed adjacent to and coupling the proximal section and the distal section, the articulating member being configured to extend within the transition region, and is located along the length of the medical device such that when the distal section is positioned adjacent the target area, the articulating section is located within the transition region.

2. The medical device of claim 1, wherein a proximal section includes a proximal shaft member having a distal end, the distal section includes a distal shaft member having a proximal end, and the articulating member includes a tube having a first end disposed over the distal end of the proximal shaft member and a second end disposed over the proximal end of the distal shaft member.

3. The medical device of claim 2, wherein the articulating member includes a plurality of slots.

4. The medical device of claim 3, wherein articulating member includes at least two slots that are adjacent with one another and include portions that overlap with each other about a circumference of the tube.

5. The medical device of claim 2, wherein the proximal shaft member includes an angled distal end.

6. The medical device of claim 2, wherein the distal shaft member includes an angled proximal end.

7. The medical device of claim 2, wherein the proximal shaft member and the distal shaft member are continuous with one another.

8. The medical device of claim 2, wherein the proximal shaft member and the distal shaft member are separated by a longitudinal space disposed adjacent the articulating section.

9. The medical device of claim 1, wherein the articulating section is tapered so as to have a reduced outside diameter relative to the proximal section.

10. The medical device of claim 1, wherein the articulating section is tapered so as to have a reduced outside diameter relative to the distal section.

11. The medical device of claim 1, further comprising a sheath disposed over at least a portion of the medical device.

12. A guidewire for accessing an intravascular treatment area in a patient, the treatment area being accessible by passing the guidewire through a first blood vessel region, through a transition region that bends at an angle of sixty degrees or greater relative to the first blood vessel region, and into a second blood vessel region, the guidewire comprising:
a proximal section having a first length adapted to extend along the first blood vessel region;
a distal section having a second length adapted to extend along the second blood vessel region to the treatment area; and
an articulating section defined by an articulating member disposed between and coupling the proximal section and the distal section, wherein the articulating section is adapted to extend within the transition region, and is located along the length of the guidewire such that when the distal section is positioned adjacent the target area, the articulating section is located within the transition region.

13. The guidewire of claim 12, wherein a proximal section includes a proximal shaft member having a distal end, the distal section includes a distal shaft member having a proximal end, and the articulating member includes a tube having a first end disposed over the distal end of the proximal shaft member and a second end disposed over the proximal end of the distal shaft member.

14. The guidewire of claim 13, wherein the articulating member includes a plurality of slots, slits, or grooves.

15. The guidewire of claim 14, wherein articulating member includes at least two slots that are adjacent with one another and include portions that overlap with each other about a circumference of the tube.

16. The guidewire of claim 13, wherein the proximal shaft member includes an angled distal end.

17. The guidewire of claim 13, wherein the distal shaft member includes an angled proximal end.

18. The guidewire of claim 13, wherein the proximal shaft member and the distal shaft member are continuous with one another.

19. The guidewire of claim 13, wherein the proximal shaft member and the distal shaft member are separated by a longitudinal space disposed adjacent the articulating section.

20. The guidewire of claim 12, wherein the articulating section is tapered so as to have a reduced outside diameter relative to the proximal section.

21. The guidewire of claim 12, wherein the articulating section is tapered so as to have a reduced outside diameter relative to the distal section.

22. The guidewire of claim 12, further comprising a sheath disposed over at least a portion of the guidewire.

23. An intracorporal guidewire for use in an anatomy of a patient, the guidewire comprising:
an elongate shaft including a proximal section and a distal section, the proximal section including a distal end and a first lateral flexibility adjacent the distal end, the distal section including a proximal end and a second lateral flexibility adjacent the proximal end; and
an articulating member disposed adjacent to and coupling the distal end of the proximal section with the proximal end of the distal section;
wherein the articulating member defines an articulating section of the shaft that is configured to extend within a bend in the anatomy of the patient that creates an angle of sixty degrees or greater, the articulating section having a greater lateral flexibility than both the first lateral flexibility and the second lateral flexibility.

24. The guidewire of claim 23, wherein the articulating member includes a tube having a first end disposed over the distal end of the proximal section and a second end disposed over the proximal end of the distal section.

25. The guidewire of claim 24, wherein the articulating member includes a plurality of slots.

26. The guidewire of claim 23, wherein the distal end of the proximal section and the proximal end of the distal section are separated by a longitudinal space, and wherein the articulating member spans the longitudinal space.

27. A guidewire, comprising:
an elongate shaft including a proximal section, a distal section, and an articulating section disposed therebetween;
the proximal section including a distal end and a first lateral flexibility adjacent the distal end, and the proximal section is configured to extend from a first position outside a patient to a second position in a first blood vessel region;
the distal section includes a proximal end and a second lateral flexibility adjacent the proximal end, and the distal section is configured to extend within a second blood vessel region to a target site; and
the articulating section is defined by an articulating member disposed adjacent to and coupling the distal end of the proximal section and proximal end of the distal section, the articulating section has a greater lateral flexibility than both the first lateral flexibility and the second lateral flexibility, and the articulating section is configured to span a transition region disposed between a first blood vessel region and a second blood vessel region wherein the second blood vessel region is oriented at an angle of sixty degrees or greater relative to the first blood vessel region.

28. A method of navigating an elongated medical device to a target site in the anatomy of a patient, the method comprising:
providing the elongated medical device including:
a proximal section including a distal end and a first lateral flexibility adjacent the distal end;
a distal section including a proximal end and a second lateral flexibility adjacent the proximal end; and
an articulating section disposed adjacent to and coupling the distal end of the proximal section with the proximal end of the distal section, the articulating section having a greater lateral flexibility than both the first lateral flexibility and the second lateral flexibility;
advancing the medical device through a first artery and into a second artery through a junction between the first artery and a second artery, the junction forming a transition region that defines a bend in the anatomy that creates an angle of sixty degrees or greater;
disposing the medical device within the anatomy such that distal section is adjacent the target site and such that the articulating section is within the transition region.

29. The method of claim 28, wherein the first artery is an abdominal aorta and the second artery is a renal artery, and the transition region is the junction between abdominal aorta and the renal artery.

30. The method of claim 28, wherein the first artery is a first femoral artery and the second artery is a second femoral artery, and the transition region is an aortic bifurcation.

31. The method of claim 28, wherein the elongated medical device comprises a guidewire.

32. A method of manufacturing a guidewire, the method comprising:
providing a proximal shaft member having a distal end and a first lateral flexibility adjacent the distal end;
providing a distal shaft member having a proximal end and a second lateral flexibility adjacent the proximal end; and
coupling the distal end of the proximal shaft member to the proximal end of the distal shaft member with a tubular articulating member, wherein coupling the distal end of the proximal shaft member to the proximal end of the distal shaft member defines an articulating region that has a lateral flexibility that is greater than both the first lateral flexibility and the second lateral flexibility.

33. The method of claim 32, wherein the guidewire is adapted for accessing an intracorporal target area in an anatomy of a patient, the anatomy of the patient including a first region and a second region connected by a transition region that forms a bend in the anatomy that creates an angle of sixty degrees or greater, and wherein the articulating section is disposed along the length of the guidewire such that when the distal section is positioned adjacent the target area, the articulating section is located within the transition region.

34. A guidewire, comprising:
an elongate shaft, the shaft including a proximal section having a distal end, and a distal section having a proximal end; and
articulating means disposed adjacent the distal end of the proximal section and adjacent the proximal end of the distal section.

35. A guidewire adapted for accessing an intracorporal target area in an anatomy of a patient, the anatomy of the patient including a first region and a second region connected by a transition region that forms a bend in the anatomy that creates an angle of sixty degrees or greater, the target area being spaced from the transition region, the guidewire comprising:
an elongate shaft including a distal end and an articulating means for extending within the transition region when the distal end is positioned adjacent the target area.

36. A guidewire, comprising:
an elongate shaft including a proximal section, a distal section, and an articulating section disposed therebetween;
the proximal section including a distal end and a first lateral flexibility adjacent the distal end;
the distal section includes a proximal end and a second lateral flexibility adjacent the proximal end; and
the articulating section being disposed between the distal end of the proximal section and proximal end of the distal section, the articulating section has a greater lateral flexibility than both the first lateral flexibility and the second lateral flexibility.

37. The guidewire of claim 36, wherein the proximal section is configured to extend from a first position outside a patient to a second position in a first blood vessel region and the distal section is configured to extend within a second blood vessel region to a target site.

38. The guidewire of claim 36, wherein the articulating section is configured to span a transition region disposed between a first blood vessel region and a second blood vessel region wherein the second blood vessel region is oriented at an angle of sixty degrees or greater relative to the first blood vessel region.

39. The guidewire of claim 36, wherein the articulating section is defined by an articulating member disposed adjacent to and coupling the distal end of the proximal section and the proximal end of the distal section.
